(19) United States
(12) Patent Application Publication
(10) Pub. No.: US 2024/0252804 A1
(43) Pub. Date: Aug. 1, 2024

(54) ANTIMICROBIAL DELIVERY DEVICES

(71) Applicant: Attilio DiFiore, West Jordan, UT (US)

(72) Inventor: Attilio DiFiore, West Jordan, UT (US)

(21) Appl. No.: 18/425,112

(22) Filed: Jan. 29, 2024

Related U.S. Application Data
(60) Provisional application No. 63/442,498, filed on Feb. 1, 2023.

(51) Int. Cl. A61M 39/16; A01N 35/10; A61M 39/20

(52) U.S. Cl. CPC A61M 39/162; A01N 35/10; A61M 39/20

(57) ABSTRACT

An antimicrobial delivery device can include a resilient porous applicator having a fluid-receiving surface and a skin contact surface, and can carry a freeze-dried antimicrobial that is deliverable to the skin contact surface when sufficient delivery fluid is introduced to the fluid-receiving surface and passes through the resilient porous applicator. A fluid channeling body can include a fluid inlet positioned at a channel fitting adapted to connect to a fluid delivery device, and can also include a fluid outlet positioned at a fluid channeling body attachment surface. The fluid channeling body attachment surface can be fixedly joined with the fluid-receiving surface of the resilient porous applicator to allow delivery fluid to flow from the fluid channeling body through a bulk of the resilient porous applicator to reconstitute the freeze-dried antimicrobial for delivery of an antimicrobial compound.

The present applications claims the benefit of U.S. Provisional Patent Application No. 63/442,498 filed Feb. 1, 2023, the entirety of which is incorporated herein by reference.

### BACKGROUND

Sponges and other applicators are used with antiseptics for preparation prior to a surgical procedure or to treat wounds in the field. For example, prior to cutting through the skin for surgery, the risk of infection can be reduced by properly sterilizing the surgical site and surrounding area. Likewise, after receiving a wound, the wound can be cleaned and treated with an antiseptic or antibiotic compound to reduce the chances of infection. Example antiseptic compounds applied to the skin prior to a surgical procedure or to clean a wound may include ethanol, isopropyl alcohol, chloroxylenol, povidone iodine, chlorhexidine gluconate or other diguanides, benzethoniuim chloride, benzalkonium chloride, antibacterial dyes (to treat burns and wounds), peroxides or permanganates, halogenated phenol derivatives, quinolone derivatives, or a combination thereof. The sponge or other applicator used to apply the antiseptic or antibiotic can be moved along the skin surface with a back and forth, circular, or other motion to treat the area for microorganisms that may be present that would otherwise lead to infection. In some instances, the application motion can provide some solution penetration through some epidermis layers, further assisting with reducing the population of skin-dwelling microorganisms that may be present that may lead to infection.

### BRIEF DESCRIPTION OF THE DRAWINGS

FIG. 1 illustrates a side cutaway view and a front perspective/partial cutaway view of an example antimicrobial delivery device in accordance with examples of the present disclosure;

FIG. 2 is a front plan/partial cutaway view of an example antimicrobial delivery device that is sterilized within a sealed container in accordance with examples of the present disclosure;

FIG. 3 is a side cutaway view of an example antimicrobial delivery device with a fluid channeling body having a small fluid channeling body volume and positioned perpendicular to a skin contact surface of a resilient porous applicator in accordance with examples of the present disclosure;

FIG. 4 is a side cutaway view of another example antimicrobial delivery device with a fluid channeling body having a small fluid channeling body volume and positioned at an acute angle relative to a skin contact surface of a resilient porous applicator in accordance with examples of the present disclosure; and

FIGS. 5-7 illustrate front perspective/partial cutaway views of three alternative example antimicrobial delivery devices in accordance with examples of the present disclosure.

### DETAILED DESCRIPTION

The present disclosure is drawn to an antimicrobial delivery device that can be used to deliver an antimicrobial solution to a skin surface of a subject, such as for surgical preparation of a skin site, disinfecting an infected wound, cleaning a wound in the field, etc. Thus, the antimicrobial delivery device can be used to treat intact skin or injured skin, e.g., wounded, cut, scraped, burned, etc.

In examples of the present disclosure, an antimicrobial delivery device can include a resilient porous applicator having a fluid-receiving surface and a skin contact surface. The resilient porous applicator can carry a freeze-dried antimicrobial that is deliverable to the skin contact surface when sufficient delivery fluid is introduced to the fluid-receiving surface and passes through the resilient porous applicator reaching the skin contact surface. The antimicrobial delivery device can likewise include a fluid channeling body including a fluid inlet and a fluid outlet. The fluid inlet can be posited at a channel fitting adapted to connect to a fluid delivery device. The fluid outlet can be positioned at a fluid channeling body attachment surface that is fixedly joined with the fluid-receiving surface of the resilient porous applicator in a manner that allows delivery fluid to flow from the fluid channeling body through a bulk of the resilient porous applicator to reconstitute the freeze-dried antimicrobial. The reconstituted freeze-dried antimicrobial can be delivered to the skin surface as an antimicrobial compound when the skin contact surface is contacted with a skin surface of a subject. In some examples, the antimicrobial delivery device may be part of an antimicrobial delivery system, which includes the antimicrobial delivery device and a fluid delivery device to introduce the delivery fluid into the antimicrobial delivery device. In another example, a method of preparing an antimicrobial delivery device can include loading a resilient porous material with an antimicrobial loading solution including a liquid carrier and from 0.5 wt % to 10 wt % of an antimicrobial compound, and freeze-drying the resilient porous material containing the loading solution to remove liquid carrier from the resilient porous material forming a resilient porous applicator loaded with a freeze-dried antimicrobial. The resilient porous applicator can include a fluid-receiving surface and a skin contact surface. The method can also include attaching the fluid-receiving surface of the resilient porous applicator to a fluid channeling body attachment surface of a fluid channeling body. The fluid channeling body can include (i) a fluid inlet positioned at a channel fitting that is connectable to a fluid delivery device, and (ii) a fluid outlet positioned at the fluid channeling body attachment surface to provide an opening for delivery fluid to enter the resilient porous applicator and reconstitute the antimicrobial compound within the resilient porous applicator.

In another example, a method of delivering an antimicrobial compound to a skin surface can include connecting a delivery device to an inlet of a fluid channeling body of an antimicrobial delivery device. The antimicrobial delivery device can further include a resilient porous applicator carrying a freeze-dried antimicrobial. The fluid channeling body can include a fluid outlet with the resilient porous applicator positioned at the fluid outlet. The method can further include flowing a delivery fluid from the delivery device through the fluid channeling body and into the resilient porous applicator to form an antimicrobial solution that includes an antimicrobial compound formed from the freeze-dried antimicrobial which is reconstituted in the delivery fluid and carried by the resilient porous applicator, and furthermore, applying the antimicrobial solution to a skin surface by applying mechanical pressure to the resilient porous applicator against the skin surface.

When discussing the antimicrobial delivery devices, antimicrobial delivery systems, or methods related thereto, such discussions can be considered applicable to one another whether or not they are explicitly discussed in the context of that example. For example, when discussing freeze-dried antimicrobial in the context of the antimicrobial delivery devices, such disclosure is also relevant to and directly supported in the context of the systems and/or methods, and vice versa.

Terms used herein will be interpreted as the ordinary meaning in the relevant technical field unless specified otherwise. In some instances, there are terms defined more specifically throughout or included at the end of the present disclosure, and thus, these terms are supplemented as having a meaning described herein.

### Antimicrobial Delivery Devices and Systems

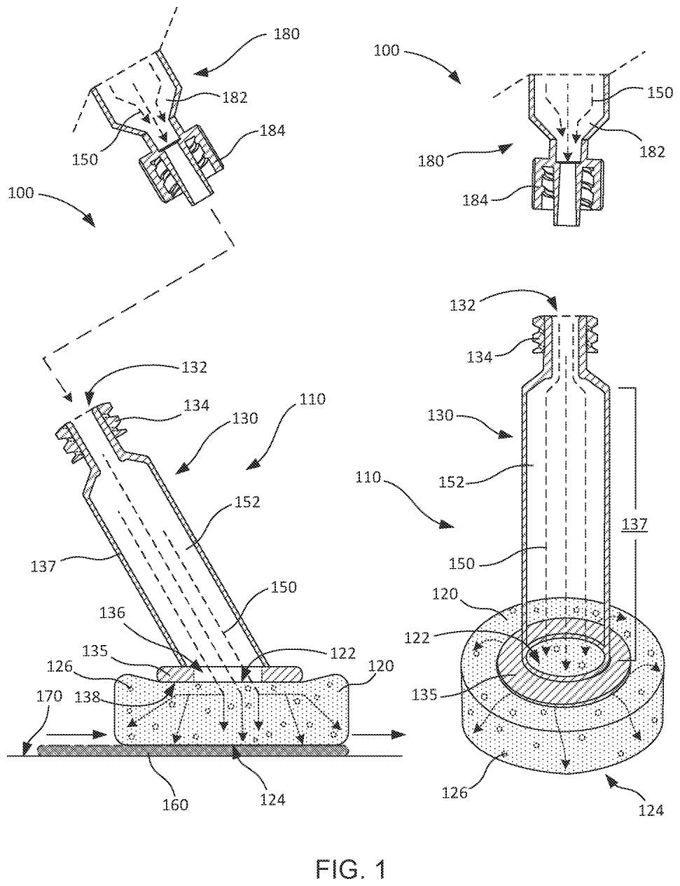

Referring now to FIG. 1, and in accordance with both a device and system example of the present disclosure, an antimicrobial delivery device 110 is shown in multiple views. Furthermore, the antimicrobial delivery device can be paired with a fluid delivery device 180 as part of an antimicrobial delivery system 100.

Referring more specifically to the antimicrobial delivery device 110, the device can include a fluid channeling body 130 that is attached to a resilient porous applicator 120. The fluid channeling body in this example is shown having a barrel 137 that is connected to a flange 135. The flange is the location of attachment of the fluid channeling body to the resilient porous applicator, whereas the barrel defines a flow path of the delivery fluid 150 through the fluid channeling body. Thus, in this example, it can be seen in the side view of the antimicrobial delivery device that the flow path of the delivery fluid exhibits an acute angle relative to the fluid channeling body attachment surface 138 of the flange. Example acute angles that can be implemented may range from about 15° to about 75°, from about 30° to about 60°, or from about 40° to about 50°.

In further detail regarding the resilient porous applicator 120, this example illustrates a fluid-receiving surface 122 and a skin contact surface 124. The resilient porous applicator may include a material such as a polymeric material (e.g., polyurethane, polyethylene, polypropylene, etc.), a fibrous material (e.g., cellulose fibers, polyethylene, polypropylene, etc.), a foam (e.g., polyurethane foam), or the like. In some examples, the resilient porous applicator may be in the structural form of a sponge, a gauze, or a layered sheet, for example. The resilient porous applicator can carry a freeze-dried antimicrobial 126 that may include, for example, an antiseptic compound, an antibiotic compound, or a combination thereof. More specific examples of freeze-dried antimicrobials that can be carried by the resilient porous applicator include an alcohol, an iodine, a chlorhexidine, a quaternary ammonium salt, an antimicrobial dye, a peroxide, a permanganate, a halogenated phenol derivative, an antimicrobial metal component, e.g., silver, copper, etc., and/or a quinolone derivative, etc. Still more specific examples of freeze-dried antimicrobials include ethanol, isopropyl alcohol, chloroxylenol, povidone iodine, chlorhexidine gluconate, chlorhexidine digluconate, chlorhexidine acetate, benzethoniuim chloride, a silver salt, colloidal silver, a silver complex, a copper component, and/or benzalkonium chloride, etc. These components and/or compounds can be used alone or in combination with one another, or in combination with other components, such as in a pre-operative or post-operative application. In a post-operative situation, other components that may not be antiseptic in nature could likewise be combined, such as hydrogels and collagen-based proteins and/or peptides involved in healing process and/or cell signaling, numbing agents such as lidocaine and/or other local anesthetics or analgesics, anti-inflammatory compounds such as non-steroidal and/or steroidal anti-inflammatory compounds, e.g. cortisone, compounds that reduce scarring, etc. Furthermore, because the antimicrobials are freeze-dried into the resilient porous applicator to be stored prior to reconstitution at the time of use, even antimicrobial compounds that might otherwise interact when admixed together can be freeze-dried together with good storage stability.

The freeze-dried antimicrobial can be deliverable to (and ultimately through) the skin contact surface 124 of the resilient porous applicator 120 when a sufficient amount of delivery fluid 150 is introduced to the fluid-receiving surface and then passes through the resilient porous applicator. The delivery fluid within the resilient porous applicator can contact and reconstitute the freeze-dried antimicrobial therein to activate or reanimate the freeze-dried antimicrobial as an antimicrobial compound effective for antimicrobial activity. Thus, the reconstituted freeze-dried antimicrobial as an antimicrobial solution 160 (carrying the antimicrobial compound) can be delivered to the skin surface 170 through the skin contact surface of the resilient porous applicator. The term “reconstitute(d)” refers to the solubilizing or suspending of the freeze-dried material that is incorporated in or absorbed by the resilient porous applicator in a manner that retains at least a portion, and in many cases the full spectrum of its pre-freeze-dried activity. The fluid channeling body 130 in this example defines a channel volume 152 including a fluid inlet 132 and a fluid outlet 136. The fluid inlet can be posited at a channel fitting 134 adapted to connect to a fluid delivery device 180 (more particularly in this example to connect to a fluid delivery fitting 184 of the fluid delivery device). The channel fitting and/or delivery device fitting can be part of a mated pair of fittings for connecting the antimicrobial delivery device to the fluid delivery device, and either or both may include a barbed connector, a luer connector, a pressure fit connector, a threaded connector, a membrane, a tube, or a combination thereof. For example, the channel fitting may be in the form of a female luer connector and the fluid delivery fitting may be in the form of a male luer connector as the mated connector pair, or vice versa. On the other end of the fluid channeling body, the fluid outlet can be positioned at a fluid channeling body attachment surface 138 that is fixedly joined with a fluid-receiving surface 122 of the resilient porous applicator 120 in a manner that allows the delivery fluid 150 to flow from the fluid channeling body through a bulk portion of the resilient porous applicator (which, in this example, is positioned between the fluid-receiving surface and the skin contact surface) to reconstitute the freeze-dried antimicrobial 126. In the example shown, the fluid channeling body attachment surface is enlarged beyond a rim of the fluid channeling body by the presence of a flange 135 that surrounds the fluid outlet. The flange in this example, provides additional surface area of attachment between the fluid channeling body and the fluid-receiving surface of the resilient porous applicator.

In some examples of the present disclosure, the resilient porous applicator can be loaded with from 1 mg to 200 mg, from 10 mg to 200 mg, from 20 mg to 100 mg, or from 80 mg to 200 mg of freeze-dried antimicrobial. With these ranges as a general non-limiting guideline, it is noted that individual antimicrobial compounds typically have a minimum inhibitory concentration (MIC) for certain predetermined pathogens, such as certain bacteria or yeast. In accordance with examples of the present disclosure, the freeze-dried antimicrobial after being reconstituted can be designed to exhibit the MIC of one or more pathogens, for example.

In some examples, the resilient porous applicator can be freeze-dried along with an antimicrobial compound so that both the resilient porous applicator is freeze-dried and the antimicrobial compound is in the form of the freeze-dried antimicrobial. In the freeze-dried state, the resilient porous applicator can have a smaller total volume compared to the total volume of the resilient porous applicator prior to being freeze-dried (or after introduction of the delivery fluid thereto). When referring to the total volume of the resilient porous applicator, the value refers to both the volume of the material as well as the void volume therein that is defined by the material surrounding the voids. In some examples, the total volume of the resilient porous applicator after freeze-drying and prior to introduction of delivery fluid thereto can be from about 0.2 cm³ to about 100 cm³, from about 1 cm³ to about 80 cm³, or from about 2 cm³ to about 60 cm³, though volumes outside of this range may be used in certain embodiments. With this in mind, it is noted that when the resilient porous applicator is loaded with a delivery fluid, the resilient porous applicator may stay at about the same volume, but in many cases, the resilient porous applicator may expand to a size up to a size prior to freeze-drying the material. The contact surface may expand laterally in some instances, but typically, most of the expansion may be in the direction of the thickness of the resilient porous applicator.

When delivery fluid is introduced to fill the resilient porous applicator and deliver the reconstituted antimicrobial compound as part of an antimicrobial solution, the total volume of the resilient porous applicator can expand in volume by about 0% to about 100%, about 2% to about 100%, or about 5% to about 50%. In some instances, the resilient porous applicator in its freeze-dried state (loaded with the freeze-dried antimicrobial) can have a moisture content of less than about 2.5 wt %. In some examples, the moisture content can range from 0.1 wt % to about 2.5 wt %, from about 0.2 wt % to about 2.5 wt %, from 0.5 wt % to about 2.5 wt %, or from 0.5 wt % to about 2 wt %. In further detail, the resilient porous applicator in its freeze-dried state (with low moisture content) can be prepared so it is capable to receive up to 3 cm³, up to 6 cm³, or even up to 10 cm³ of delivery fluid without allowing the delivery fluid to escape from the resilient porous applicator, e.g., when the delivery fluid is introduced to the fluid-receiving surface causing the resilient porous applicator to partially or fully fill up and is not otherwise mechanically compressed or agitated in a manner to release delivery fluid or antimicrobial solution formed therefrom.

In additional detail, the resilient porous applicator 120 can be sized and positioned relative to the fluid outlet 136 so that delivery of 3 cm³ or less of a delivery fluid is sufficient to contact from 95 wt % to 100 wt % of the freeze-dried antimicrobial carried by the resilient porous applicator. In other examples, the resilient porous applicator is sized and positioned relative to the outlet so that delivery of 12 cm³ or less of a delivery fluid is sufficient to contact from 95 wt % to 100 wt % of the freeze-dried antimicrobial carried by the resilient porous applicator. In still other examples, the resilient porous applicator is sized and positioned relative to the outlet so that delivery of 30 cm³ or less of a delivery fluid is sufficient to contact from 95 wt % to 100 wt % of the freeze-dried antimicrobial carried by the resilient porous applicator. This can occur as the delivery fluid 150 is received by the resilient porous applicator and wicks from this location to from most to all of the resilient porous applicator.

Turning now to the antimicrobial skin-delivery system 100 also shown by way of example at FIG. 1, the system includes the antimicrobial delivery device as described above and elsewhere herein, but also includes a fluid delivery device 180. The fluid delivery device in this example includes a fluid delivery body 182 that defines a delivery device volume 186. The fluid delivery device also includes a fluid delivery fitting 184, which typically would be configured to connect to the channel fitting of the fluid channeling body 130 of the antimicrobial delivery device 110, as described previously.

It is noted that with respect to the antimicrobial delivery device 110, the fluid channeling body 130 may be configured to contain any of a number of fluid volumes. For example, the fluid channeling body may have a channel volume 152 ranging from 0.1 cm³ to 10 cm³, from 0.5 cm³ to 10 cm³, from 1 cm³ to 5 cm³, or from 2 cm³ to 8 cm³, for example. With this in mind, as getting the delivery fluid 150 into the resilient porous applicator 120 to reconstitute the freeze-dried antimicrobial 126 therein, it is understood that any volume introduced into the antimicrobial delivery device 110 should be calculated based on the amount of delivery fluid that actually reaches the resilient porous applicator when loading the antimicrobial delivery device. For example, there may be unused delivery fluid that remains in the channel volume of the fluid channeling body once the resilient porous applicator is loaded. As a result, providing an antimicrobial delivery device with smaller channel volumes would thus call for the introduction of smaller volumes of delivery fluid, as a higher percentage of the delivery fluid would reach the resilient porous applicator compared to devices having larger channel volumes. By way of specific example, if the resilient porous applicator is to receive 3 cm³ of delivery fluid for operation, and the fluid channeling body is 3 cm³ in volume, the total of 6 cm³ of delivery fluid may be introduced into the antimicrobial delivery device from the fluid delivery device in preparation for use.

In further detail and by way of example, the amount of freeze-dried antimicrobial in the resilient porous applicator can be such that by introducing 3 cm³ of delivery fluid into the resilient porous applicator (through the fluid channeling body), a 1 wt % to 5 wt % solution of antimicrobial compound can be generated. In another example, the amount of freeze-dried antimicrobial in the resilient porous applicator can be such that by introducing 1 cm³ of delivery fluid into the resilient porous applicator (through the fluid channeling body), a 0.5 wt % to 3 wt % solution of antimicrobial compound can be generated. In another example, the amount of freeze-dried antimicrobial in the resilient porous applicator can be such that by introducing 10 cm³ of delivery fluid into the resilient porous applicator (through the fluid channeling body), a 2 wt % to 8 wt % solution of antimicrobial compound can be generated. These amounts refer to the amount of delivery fluid that reaches the resilient porous applicator, and not to the total amount of delivery fluid introduced from the fluid delivery device, a portion of which may remain or be discarded from the fluid channeling body when in use. Furthermore, it is noted that the weight percent of antimicrobial compound in the solution is based on the freeze-dried antimicrobial after being reconstituted by the delivery fluid, and thus excludes the weight of the porous material.

In further detail regarding the antimicrobial delivery system, the fluid delivery device may be in the form of any of a number of devices, such as a syringe, an ampoule, a fluid pump, an automated fluid metering device, an IV bag, etc. In some examples, it is noted that the antimicrobial delivery system may include the delivery fluid as part of the system. The delivery fluid may be an aqueous liquid, an alcohol, a peroxide, saline, sterile water, ethanol, alcohol, or a combination thereof.

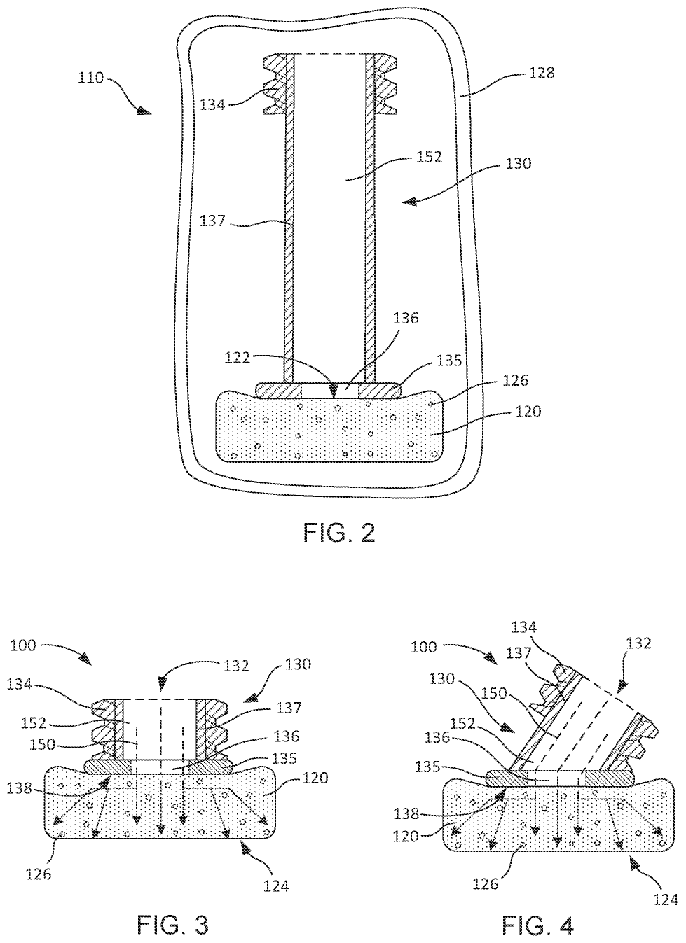

Referring now to FIG. 2, a front plan/partial cutaway view of an example antimicrobial delivery device 110 is shown that is similar to that of FIG. 1. Some of the features shown in FIG. 1 are illustrated by example here, including a fluid channeling body 130 that includes a channel fitting 134 and a flange 135, and a channel volume 152 for the passing of delivery fluid (not shown). Delivery fluid introduced into the channel volume is passed through a fluid outlet and into a resilient porous applicator 120, which carries freeze-dried antimicrobial therein. In this particular example, a sealed container 128 or bag is shown to illustrate how the antimicrobial delivery device may be manufactured, shipped, and stored in order to maintain the sterility of the antimicrobial device prior to use.

Turning now to FIG. 3 and FIG. 4, it is noted that both of these examples present schematic side cutaway views of two different antimicrobial delivery devices. FIG. 3 illustrates an antimicrobial delivery device 110 having a flow path of delivery fluid 150 that exhibits a right angle relative to the fluid channeling body attachment surface 138 of the flange 135. FIG. 4, on the other hand, illustrates an antimicrobial delivery device 110 having a flow path of delivery fluid 150 that exhibits about a 45° angle relative to the fluid channeling body attachment surface 138 of the flange 135. Both of these structures include similar structures as those described in FIG. 1, however, in these examples, the channel volumes 152 are more minimized, e.g., contained within about the length of the channel fittings 134. By shortening the length of the channel volume (or otherwise reducing the volume of the channel volume), the amount of delivery fluid that is used which is not received by the resilient porous applicator 120 during use can be minimized.

In further detail with respect to FIGS. 3 and 4, many of the same structures shown in FIG. 1 are shown by way of example in these examples as well. Referring to both FIGS. 3 and 4 collectively, both antimicrobial delivery devices 110 include a resilient porous applicator 120 with a fluid-receiving surface 122 and a skin contact surface 124. The resilient porous applicator is loaded in these examples with a freeze-dried antimicrobial. The resilient porous applicator is attached at its fluid-receiving surface to a fluid channeling body attachment surface 138 of a fluid channeling body 130. In this particular instance, the fluid channeling body attachment surface is associated with a flange 135 that provides increased surface area for attachment to the resilient porous applicator. In further detail, the fluid channeling body includes a barrel 137 to carry the delivery fluid 150 and a channel fitting 134 at or about a fluid inlet 132. The other end of the fluid channeling body includes a fluid outlet 136, which in this example is located adjacent to the flange. Thus, the delivery fluid is introduced into a channel volume 152 of the fluid channeling body at the fluid inlet and then exits the fluid channeling body at the fluid outlet, thus introducing the delivery fluid into the resilient porous applicator.

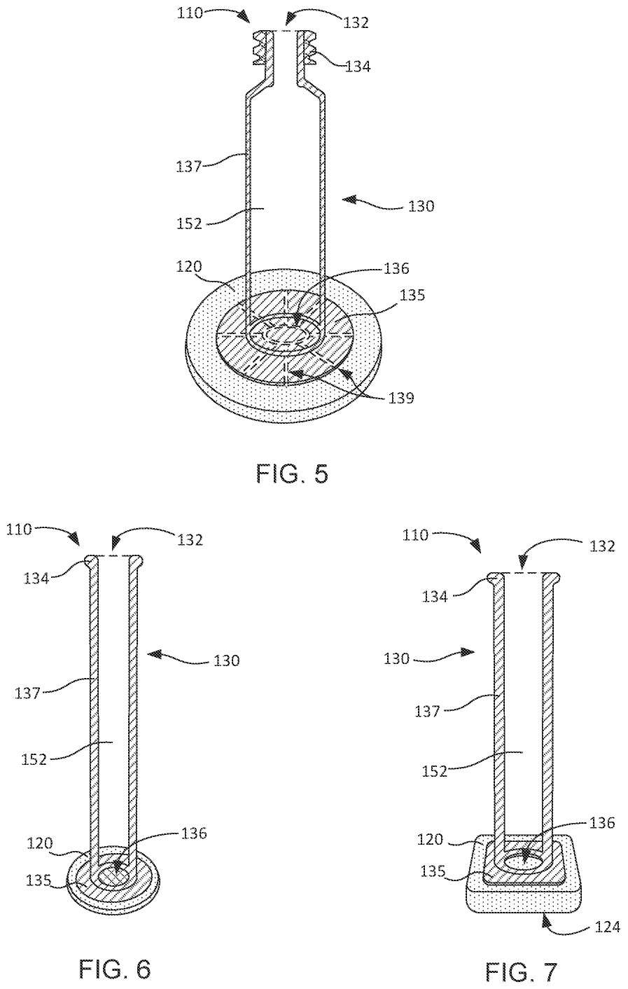

FIGS. 5-7 illustrate front perspective/partial cutaway views of three alternative example antimicrobial delivery devices 110 in accordance with examples of the present disclosure. In each instance, the antimicrobial device includes a resilient porous applicator 120 that is attached to a fluid channeling body 130. The resilient porous applicator would contain a freeze-dried antimicrobial (not show) as previously described. In further detail, the fluid channeling body includes a fluid inlet 132 and a fluid outlet 136. A channeling fitting 134, such as a luer connector, is positioned at or about the fluid inlet and a flange 135 is positioned at or about the fluid outlet. Furthermore, a barrel 137 is positioned between the flange and the channel fitting to carry delivery fluid therebetween. Other features may also be included as shown by way of example in FIGS. 1-4.

Regarding FIG. 5 in particular, the flange 135 is designed such that the fluid outlet includes a plurality of flow paths 139 to channel delivery fluid from the barrel 137 into the resilient porous applicator. The plurality of flow paths in this example are shown as radiating out within the flange, but there are many other configurations that can be used to distribute delivery fluid. Some examples may include the presence of flow paths that i) tunnel through the flange more directly from the channel volume of the barrel and into the resilient porous applicator, ii) provide overpasses or underpasses relative to other flow paths, iii) provide wider flow paths to accommodate more delivery fluid for faster resilient porous applicator loading, etc. In other examples, there may be a central fluid outlet (similar to that shown in FIGS. 1-4) in addition to the plurality of flow paths shown in this example. This would allow for central delivery fluid loading to the resilient porous applicator and to more lateral locations thereof.

Regarding FIG. 6, a more elongated barrel 137 of the fluid channeling body 130 is shown relative to the footprint area of the resilient porous applicator 120. Furthermore, in this example, the flange 135 of the fluid channeling body is shown as being embedded within a recess of the resilient porous applicator. Notably, the thickness of the flange is not shown in this example, as it is embedded within the resilient porous applicator. Furthermore, in this example, at the fluid outlet 136, there is a different material present compared to the material used for the resilient porous applicator. The different material can be used to assist with wicking of the delivery fluid in a more controlled manner, to assist with the spreading of the delivery fluid, etc. In other words, this different material can be used to modulate or control in some manner the distribution of the delivery fluid to various locations of the resilient porous applicator, which may be comparable to the manner in which the plurality of flow paths shown in FIG. 5 may mechanically control the distribution of delivery fluid. Though not shown, in some examples, the fluid outlet could be designed to include the plurality of flow paths (as shown in FIG. 5) in combination with the presence of a different material at the fluid outlet (as shown in FIG. 6).

Referring now more specifically to FIG. 7, this example illustrates that there may be different resilient porous applicator 120 configurations. In this instance, the resilient porous applicator has a rectangular shape at the skin contact surface 124. It is also notable that the channel fitting 134 shown in this example and in the FIG. 6 example has a simpler configuration than that shown in FIGS. 1-5. The channel fitting can thus be of any configuration suitable for attaching a fluid delivery device (not shown in this example, but shown in FIG. 1) thereto.

### Methods of Preparing Antimicrobial Delivery Devices

In accordance with examples of the present disclosure, a method of preparing an antimicrobial delivery device can include loading a resilient porous material with an antimicrobial loading solution including a liquid carrier and from 0.5 wt % to 10 wt % of an antimicrobial compound, and freeze-drying the resilient porous material containing the antimicrobial loading solution to remove liquid carrier from the resilient porous material forming a resilient porous applicator loaded with a freeze-dried antimicrobial. The resilient porous applicator can include a fluid-receiving surface and a skin contacting surface. The method can also include attaching the fluid-receiving surface of the resilient porous applicator to a fluid channeling body attachment surface of a fluid channeling body. The fluid channeling body can include (i) a fluid inlet positioned at a channel fitting that is connectable to a fluid delivery device, and (ii) a fluid outlet positioned at the fluid channeling body attachment surface to provide an opening for delivery fluid to enter the resilient porous applicator and reconstitute, e.g., solubilize and/or suspend, the antimicrobial compound within the resilient porous applicator. In more specific detail, freeze-drying can occur under vacuum pressure of less than about 100 mTorr and includes a sublimation period ranging from about −45° C. to less than about 10° C. followed by an evaporation period ranging from about 0° ° C. to about 25° C. In some examples, from about 70 wt % to about 90 wt % of the freeze-dried antimicrobial may be formed during the sublimation period, and about 10 wt % to about 30 wt % of the freeze-dried antimicrobial may be formed during the evaporation period. In other examples, the resilient porous applicator with freeze-dried antimicrobial therein prior to introduction of the delivery fluid can have a total volume of about 0.2 cm³ to about 100 cm³ (total volume includes both material volume and void volume defined by the material).

The method of preparing the antimicrobial delivery device can utilize any of the structures, components, processes, materials, etc., in the context of any of the antimicrobial delivery devices and systems described herein. However, in some more specific examples, the resilient porous applicator can have a moisture content of less than about 2.5 wt %. In other examples, the resilient porous applicator is loaded with from 10 mg to 200 mg of the freeze-dried antimicrobial. The channel fitting may include, for example, a barbed connector, a luer connector, a pressure fit connector, a threaded connector, a membrane, a tube, or a combination thereof. The method can also include sterilizing the antimicrobial delivery device and sealing the antimicrobial skin delivery device in a container. The resilient porous applicator includes a polymeric material, a fibrous material, or a solidified foam. The freeze-dried antimicrobial can include, for example, an antiseptic compound and/or an antibiotic compound. Example freeze-dried antimicrobials that can be used include an iodine, a chlorhexidine, a quaternary ammonium salt, an antimicrobial dye, a peroxide, a permanganate, a halogenated phenol derivative, a quinolone derivative, or a combination thereof.

When preparing the resilient porous applicator, there are typically two preparation stages, each having their own parameters, materials choices, part configurations, formulations, etc., that can be adjusted to achieved different results. The two preparation stages include the loading stage where an antimicrobial loading solution is used to saturate or soak into a resilient porous material, and then there is a freeze-drying stage, where to resilient porous material loaded with the antimicrobial loading solution is freeze-dried to remove most of the liquid carrier, leaving behind a freeze-dried antimicrobial therein.

The loading stage can be carried out using various solution concentrations of antimicrobial compound, various antimicrobial compounds, various types of resilient porous material, various loading periods (time) in which the resilient porous material is in contact with the antimicrobial loading solution prior to removal and being placed in the freeze-drying device.

Example antimicrobial compound concentrations in the antimicrobial loading solution may range from about 0.1 wt % to about 20 wt %, from about 0.5 wt % to about 15 wt %, from about 0.5 wt % to about 10 wt %, or from about 0.5 wt % to about 5 wt %, from about 1 wt % to about 10 wt %, or from about 1 wt % to about 5 wt %, for example. Example antimicrobial compounds that can be included in the antimicrobial loading solution include, for example, antiseptics and/or antibiotics. More specific classes of antimicrobial compounds that can be present include an alcohol, an iodine, a chlorhexidine, a quaternary ammonium salt, an antimicrobial dye, a peroxide, a permanganate, a halogenated phenol derivative, and/or a quinolone derivative, etc. More specific examples include ethanol, isopropyl alcohol, chloroxylenol, povidone iodine, chlorhexidine gluconate, chlorhexidine digluconate, chlorhexidine acetate, benzethoniuim chloride, and/or benzalkonium chloride, etc. The balance of the antimicrobial loading solution may include water, e.g., deionized water, or a mixture of water and water miscible organic solvent, e.g., alcohol, among others.

Example resilient porous materials that can be used include any of a number of polymeric material (e.g., polyurethane, polyethylene, polypropylene, etc.), a fibrous material (e.g., cellulose fibers, polyethylene, polypropylene, etc.), a foam (e.g., polyurethane foam), or the like. The resilient porous material is selected with pours so that it can hold the antimicrobial loading solution for subsequent freeze-drying, and may also be selected to be soft so that it may contact the skin surface, e.g., rubbing, pushing, compressing, etc., while not damaging or scraping the skin during use. Thus, the term “soft” is defined herein to include materials that are resilient when compressed, but furthermore, would also not scratch the skin when a wet sample of the material is rubbed and compressed along the skin during a normal antiseptic application process, such as in preparation for surgery. Soft materials may be particularly useful when applying to damaged skin or wounds, e.g., broken or burned skin. Notably, there are some porous materials, such as hard ceramics, pumice stones, etc. that would not be considered to be “soft” or even “resilient,” though these materials would be sufficiently porous to receive and hold antimicrobial loading solution. In one example, the resilient porous material may also be considered to be a “soft” material, such as a polyurethane sponge or polyurethane foam, e.g., Rynell 562B medical grade foam. The size of the resilient porous material selected for loading can be any size appropriate for application of an antimicrobial compound to a skin surface. Or, the resilient porous material can have a larger size and after loading and freeze-drying, the loaded resilient porous material can be cut down to size in the form of individual resilient porous applicators, e.g., larger sheets of material can be cut into smaller shapes for use on individual antimicrobial delivery devices.

A resilient porous applicator can be prepared using sheets of material that are cut down to size before or after loading and freeze-drying, or can be prepared from material manufactured at the appropriate size, for example. For example, a 12 inches by 24 inches sheet of polyurethane foam having about 10 mm thickness when loaded with an aqueous-based antimicrobial solution may shrink when freeze-dried to an about 9 inches by 18 inches sheet of freeze-dried polyurethane foam loaded with freeze-dried antimicrobial. The sheet may then be cut into square, round, oval, or other shaped resilient porous applicators and then individually attached to a fluid channeling body as described herein. When the freeze-dried resilient porous applicator receives a delivery fluid from the fluid channeling body, the foam may expand back to about its original proportional size in thickness, but may be constrained to some degree from fully expanding in the X- and/or Y-directions because of its attachment to the fluid channeling body. By way of one example, certain polyurethane foams can expand about 50-60% by volume from their freeze-dried state to when delivery fluid is introduced therein to reconstitute the antimicrobial compound in preparation for application t a skin surface.

With this background, a resilient porous applicator after freeze-drying with an antimicrobial compound can have X- and/or Y-direction dimensions independently from about 1 cm to about 20 cm, from about 2 cm to about 10 cm, or rom about 2 cm to about 6 cm. The shapes at the skin contact surface can thus be square, rectangular, round, oval, or the like. However, other shapes can be used, including such shapes as triangle, hexagon, parallelogram, rhombus, or other geometric configurations. Regarding thicknesses, the resilient porous applicator in the Z-direction may range from about 1 mm to about 40 mm, from about 2 mm to about 20 mm, from about 2 mm to about 15 mm, from about 1 mm to about 10 mm, or from about 2 mm to about 10 mm, for example.

The antimicrobial loading solution can be soaked or saturated into the resilient porous material by soaking, spraying, dripping, or by any other technique used to load liquid into an absorbent material. When soaking, for example, soak times can contribute to how much of the antimicrobial loading solution enters the resilient porous material. A quick dip may not fully saturate the resilient porous material, whereas a lengthy soak may not be needed to fully saturate the resilient porous material. Depending on the material choice and surface area vs. volume ratio, soak periods or times can be determined by experimentation. After loading the resilient porous material with the antimicrobial loading solution (which includes the antimicrobial compound), often the resilient porous material will be in a swollen state. This swollen state may be reversed upon freeze-drying as the liquid component is removed from the resilient porous material, leaving a resilient porous applicator containing the freeze-dried antimicrobial.

The second stage, after loading the resilient porous material with the antimicrobial loading solution, is the freeze-drying stage. The freeze-drying stage can be carried out using various parameters, including temperature ranges, temperature ramping profiles, hold times, vacuum pressures, etc. Temperatures can typically range from about −40° C. to about 30° C., from about −35° C. to about 25° C., or from about −30° C. to about 25° C. Ramping profiles may include starting at the bottom end of the temperature range and ramping that temperature up over a period of at least 12 hours, at least 18 hours, at least 24 hours, at least 36 hours, or at least 48 hours. During the ramp up of temperature, there may be periods of time where the temperature is held, e.g., hold times. Hold times may occur to complete sublimation prior to raising the temperature from sublimations temperatures to evaporation temperatures. More typically, hold times may occur during or at the end of the evaporation temperature time frame, allowing the resilient porous applicator being formed to be completed at or below a desired moisture content, e.g., less than about 2.5 wt % moisture content. A resilient porous applicator with a lower moisture content will be more likely to draw delivery fluid from the fluid channeling body into the resilient porous applicator (to reconstitute the freeze-dried antimicrobial and form an antimicrobial solution for delivery to a skin surface). Hold times may range from 5 minutes to 8 hours, from 10 minutes to 4 hours, or from 30 minutes to 2 hours, for example. During the freeze-drying process, typically the freeze-drying chamber is held at vacuum pressures. Typically, lower pressures can be desirable, but for practical purposes, the vacuum pressure can range from about 0 mTorr to 750 mTorr, from 0 mTorr to 500 mTorr, from 10 mTorr to 600 mTorr, from 50 mTorr to 500 mTorr, or from 100 mTorr to 450 mTorr, for example.

After freeze-drying, a resilient porous applicator is formed that may be about the same size as the resilient porous material prior to loading the antimicrobial loading solution, but is still resilient, compressible, and typically soft enough for comfortable application when rubbing across and/or compressing against the skin surface of a subject to apply the antimicrobial solution thereto. As a note, another approach to preparing the resilient porous applicators described herein would be to prepare a larger resilient porous sheet or mass from a large resilient porous material, and then after the larger resilient porous sheet or mass is loaded and freeze-dried, it could then be cut to size, forming a plurality of individual resilient porous applicators within the size ranges described previously.

Regardless of whether the resilient porous applicator is sized prior to loading the antimicrobial loading solution, or after freeze-drying, individual resilient porous applicators are now ready to be attached to fluid channeling bodies to assemble the antimicrobial delivery devices of the present disclosure. For example, a flange at a delivery end of a fluid channeling body can be attached to a fluid-receiving surface of the resilient porous applicator using an adhesive compound, a two-part adhesive compound, a UV curing adhesive compound, or the like.

### Methods of Delivering Antimicrobial Compounds to Skin Surfaces

Methods of delivering an antimicrobial compound to skin surfaces can include, for example, connecting a delivery device, e.g., syringe, ampoule, fluid pump, automated fluid metering device, etc., to an inlet of a fluid channeling body of an antimicrobial delivery device. The antimicrobial delivery device can further include a resilient porous applicator carrying a freeze-dried antimicrobial. The fluid channeling body can include a fluid outlet with the resilient porous applicator positioned at the fluid outlet. The method can further include flowing a delivery fluid from the delivery device through the fluid channeling body and into the resilient porous applicator to form an antimicrobial solution that includes an antimicrobial compound formed from the freeze-dried antimicrobial which is reconstituted in the delivery fluid and carried by the resilient porous applicator, and furthermore, applying the antimicrobial solution to a skin surface by applying mechanical pressure to the resilient porous applicator against the skin surface. Connecting the delivery device to the fluid channeling body can be via a pair of mated luer connectors or barbed connectors. Other mechanical connections can likewise be used, such as pressure fit connections, connections via puncture of membranes, etc. In some examples, the fluid channeling body can be used as a handle when applying the antimicrobial solution to the skin surface. For example, the fluid channeling body may include an elongated barrel that can be used as the handle. In other examples, the antimicrobial solution formed in the resilient porous applicator can include from 0.5 wt % to 10 wt % or from 1 wt % to 5 wt % of the antimicrobial compound. Flowing the delivery fluid of this method can include flowing from 1 cm³ to 20 cm³ or from 3 cm³ to 10 cm³ of delivery fluid into the resilient porous applicator.

The method of delivering the antimicrobial compound to the skin surface can utilize any of the structures, components, processes, materials, etc., in the context of any of the antimicrobial delivery devices and systems described herein. However, in some more specific detail, the freeze-dried antimicrobial may include, for example, an antiseptic compound and/or an antibiotic compound. Furthermore, the freeze-dried antimicrobial may include an alcohol, an iodine, a chlorhexidine, a quaternary ammonium salt, an antimicrobial dye, a peroxide, a permanganate, a halogenated phenol derivative, a quinolone derivative, or a combination thereof. In still other examples, the resilient porous applicator can be in the form of a sponge and the mechanical pressure may include applying a back and forth motion to the sponge against the skin surface, compressing the sponge against the skin surface, or both.

In further detail, it is noted that the antimicrobial solution delivered from the resilient porous applicator to the skin surface can be for any of a number or purposes, such as preparing a skin site for surgery (e.g., to avoid infection at surgical site), preparing a skin site for injection or puncture (e.g., catheters, IV infusions, blood draws, epidurals, shunts, etc.), treating injured skin (e.g., cuts, gashes, scrapes, burns, etc.), or treating an existing skin infection (e.g., acne, warts, lesions, cold sores, cellulitis, folliculitis, ringworm, thrush, onychomycosis, parasitic infections, chickenpox, hand foot and mouth disease, etc.).

To delivery antimicrobial solution to a skin surface in accordance with the present disclosure, a fluid delivery device, such as a syringe or ampoule, is connected to a channel fitting 134 of a fluid channeling body shown at 130 in FIGS. 1-7. The fluid delivery device may, for example, include a male luer nozzle that connects with a female luer connector of the fluid channeling body. Once connected, a delivery fluid is injected into the fluid channeling body where it ultimately is absorbed into the resilient porous applicator. Some of the delivery fluid may be forced into the resilient porous applicator, but that is required. The delivery fluid may simply be pushed into the channel volume of the fluid channeling body and then the resilient porous applicator absorbs the delivery fluid therein through a fluid outlet of the fluid channeling body. Thus, some delivery fluid may be forced into the resilient porous applicator and some may be absorbed therein, or the delivery fluid may be injected into the fluid channeling body where it is all absorbed into the resilient porous applicator. Either way, once the delivery fluid enters the resilient fluid applicator, there it reconstitutes the freeze-dried antimicrobial to reconstitute the antimicrobial to a reconstituted antimicrobial compound for delivery with the delivery fluid to a skin surface as an antimicrobial solution. The amount of delivery fluid injected from the fluid delivery device into the antimicrobial delivery device may depend on various factors, including the absorptive properties and/or size of the resilient porous applicator, the volume of the fluid channeling body, the volume of delivery fluid the resilient porous applicator can receive before saturate, the desired concentration of antimicrobial compound to be delivered to the sink surface, configuration of the fluid outlet where delivery fluid passes from the fluid channeling body into the resilient porous applicator, etc. These features can be adjusted as desired to achieve a desired result. However, in many examples, delivery fluid can be loaded in the fluid delivery device for injection into the antimicrobial delivery device at from about 1 cc to about 30 cc, from about 2 cc to about 15 cc, from about 3 cc to about 10 cc, or from about 3 cc to about 8 cc.

### Definitions

It is to be noted that, as used in this specification and the appended claims, the singular forms of “a,” “an,” and “the” include plural referents unless the context clearly dictates otherwise.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the description herein.

The terms “fitting” or the like refer to the connection portion of two or more volumes at the location where they are joined together. For example, one type of connector is referred to as a luer connector or a luer fitting. These connectors typically include a female luer fitting and a male luer fitting, e.g., a female luer fitting at the end or along an antimicrobial delivery device as described herein and a male luer nozzle at the end or along a fluid delivery or extraction device (or vice versa). The female and male portion together form a “connection.” Other types of fittings included barbed fittings (where one device includes exterior barbs and the other device may or may not include corresponding interior structures designed to receive the barbs. Other types of fittings may include pressure fit connectors, threaded connectors, a needle corresponding to an opening or membrane to be punctured, etc.

The term “fluid channeling body” refers to any structure that can be used to channel a delivery fluid from a delivery device to a resilient porous applicator. The fluid channeling body can include, for example, a channel volume defined by a barrel, a tube, multiple channels, or volumes of other shapes or configurations.

The term “antimicrobial” is used to refer to antiseptics and/or antibiotics that can be used to disinfect a skin surface of microbes, including the removal or killing of infectious bacterial, fungal, viral, and/or other similar pathogens. Antimicrobials can be in the form of freeze-dried antimicrobials or as antimicrobial compounds. The freeze-dried antimicrobials include those that have been lyophilized and are carried by a resilient porous applicator, whereas antimicrobial compounds include those antimicrobials prior to freeze-drying or after being reconstituted from their freeze-dried state.

The term “antimicrobial loading solution” refers to the solution of dispersion of liquid carrier and antimicrobial compound that is used to load the resilient porous material prior to freeze-drying.

The term “delivery fluid” refers to any liquid or liquid suspension that can be injected into the antimicrobial delivery devices of the present disclosure for the purpose of reconstituting freeze-dried antimicrobials within the resilient porous applicator to form an antimicrobial solution for delivering antimicrobial compounds to a skin surface.

The term “antimicrobial solution” refers to solutions or dispersions of antimicrobial compounds that have been reconstituted by a delivery fluid within a resilient porous applicator. The antimicrobial solution is ultimately what is delivered to the skin surface of the subject.

The term “skin” or “skin surface” refers to any surface of any animal that has skin, including humans and other mammals. The skin surface may be intact skin, wounded or broken skin, mucosal surfaces (e.g., oral, nasal, anal, vaginal, etc.), or the like.

As used herein, a plurality of items, structural elements, compositional components, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Dimensions, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also the individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

### EXAMPLES

### Example 1—Preparation of Resilient Porous Applicator

A polyurethane foam sponge (Rynell 562B) having about a 2.5 cm circular disk shape in the X- and Y-directions and about 10 mm thickness in the Z-direction is placed in a bath of an antimicrobial loading solution (2 wt % chlorhexidine gluconate and 98 wt % deionized water) until the sponge is fully saturated with the antimicrobial loading solution. The sponge loaded with the antimicrobial loading solution is carefully removed from the bath and placed in a freeze-drying chamber. Notably, the sponge is swollen with liquid compared to the original size of the sponge prior to loading. Once in the freeze-drying chamber, vacuum (negative) pressure is applied to about 400 mTorr and the temperature is dropped to about −30° C. Over a period of about 24 hours, the temperature is slowly ramped up from about −30° C. to about 25° C. (or Room Temperature). Notably, when the product temperature stays below the glass transition temperature, this is usually when the process is in a sublimation state, and when the product temperature goes above the glass transition temperature, it is typically in an evaporation state. With some materials and chemistries, as long as the material is frozen, it can be in a sublimating state.

These temperature ranges can be modified based on various factors, but these ranges represent an estimate of what is occurring during the freeze-drying process. Hold time(s) may be implemented within the freeze-drying chamber to achieve certain effects. For example, once room temperature reached, evaporation may be allowed to continue. When the tracings measured at room temperature hit a steady state, a hold time of about 2 to 4 hours is carried out so that secondary drying will be considered sufficiently complete prior to removing the resilient porous applicator from the chamber. Hold times may be implemented to achieve desired moisture content in the resilient porous applicator, e.g., less than about 2.5 wt % moisture content. When the freeze-drying cycle is complete, any remaining vacuum pressure is released resulting in ambient pressure and the resilient porous applicator is removed. In this example, the resilient porous applicator is estimated to be loaded with about 180 to 200 mg of freeze-dried chlorhexidine. The size of the sponge after freeze-drying is about the same size as the sponge prior to becoming swollen due to saturation with the antimicrobial loading solution.

### Example 2—Assembling Antimicrobial Delivery Device

The disk-shaped resilient porous applicator prepared in accordance with Example 1 is attached to a fluid channeling body to form the antimicrobial delivery device. The fluid channeling body includes i) a female luer connector at one end positioned about a fluid inlet and ii) a flange at the other end positioned about a fluid outlet. A barrel having a channel volume of about 3 cm³ is positioned between the fluid inlet and the fluid outlet. The resilient porous applicator includes a fluid-receiving surface where fluid is received from the fluid channeling body and also where the resilient porous applicator is attached to the fluid channeling body. The resilient porous applicator also includes a skin contact surface where application of the antimicrobial solution occurs after delivery fluid is passed through the resilient porous applicator and picks up antimicrobial compound that was freeze-dried therein. More specifically, the flange of the fluid channeling body is what is attached to the fluid-receiving surface. Attachment is carried out using an adhesive, e.g., acrylic adhesive, urethane adhesive, or UV adhesive; or ultrasonic welding to create a melt between the two materials. Thus, the antimicrobial delivery device now includes a fluid channeling body fixedly attached to a resilient porous applicator, and the resilient porous applicator includes the freeze-dried antimicrobial carried therein.

### Example 3—Using Antimicrobial Delivery Device to Apply Antimicrobial Solution to a Skin Surface

In preparation for surgery (or to otherwise treat a skin surface, e.g., acne treatment, wound cleaning, etc.), a syringe with a male luer nozzle is loaded with 5 cc of a delivery fluid. The delivery fluid included sterile saline. The male luer nozzle is then connected to the female luer connector of the antimicrobial delivery device of Example 2. Next, the 5 cc of delivery fluid is slowly plunged into the antimicrobial delivery device. Initially, 2 cc of delivery fluid passes through the fluid channeling body and is forced into the resilient porous applicator and the remainder of the 5 cc delivery fluid, i.e. 3 cc delivery fluid, enters the channel volume as a reservoir of delivery fluid to wick into the resilient porous applicator over time, or in some instances as the antimicrobial solution is applied to the skin surface causing the resilient porous applicator to draw additional delivery fluid therein. As the delivery fluid passes through the resilient porous applicator, the delivery fluid picks up the freeze-dried antimicrobial therein and forms an antimicrobial solution containing a reconstituted antimicrobial compound.

1. An antimicrobial delivery device, comprising:
a resilient porous applicator having a fluid-receiving surface and a skin contact surface, wherein the resilient porous applicator carries a freeze-dried antimicrobial that is deliverable to the skin contact surface when sufficient delivery fluid is introduced to the fluid-receiving surface and passes through the resilient porous applicator reaching the skin contact surface; and
a fluid channeling body including a fluid inlet and a fluid outlet, the fluid inlet positioned at a channel fitting adapted to connect to a fluid delivery device and the fluid outlet positioned at a fluid channeling body attachment surface, wherein the fluid channeling body attachment surface is fixedly joined with the fluid-receiving surface of the resilient porous applicator to allow delivery fluid to flow from the fluid channeling body through a bulk of the resilient porous applicator to reconstitute the freeze-dried antimicrobial for delivery of an antimicrobial compound from the skin contact surface to a skin surface of a subject.

2. The antimicrobial delivery device of claim 1, wherein the channel fitting includes a barbed connector, a luer connector, a pressure fit connector, a threaded connector, a membrane, a tube, or a combination thereof.

3. The antimicrobial delivery device of claim 1, wherein the resilient porous applicator:
includes a polymeric material, a fibrous material, or a solidified foam;
is in the form of a sponge a gauze, or a wipe; or
both.

4. (canceled)

5. The antimicrobial delivery device of claim 1, wherein the freeze-dried antimicrobial includes an antiseptic compound or an antibiotic compound.

6. (canceled)

7. The antimicrobial delivery device of claim 1, wherein the freeze-dried antimicrobial includes an alcohol, an iodine, a chlorhexidine, a quaternary ammonium salt, an antimicrobial dye, a peroxide, a permanganate, a halogenated phenol derivative, a quinolone derivative, or a combination thereof.

8-10. (canceled)

11. The antimicrobial delivery device of claim 1, wherein the resilient porous applicator is:
sized and positioned relative to the outlet so that delivery of 3 cm³ or less of a delivery fluid is sufficient to contact from 95 wt % to 100 wt % of the freeze-dried antimicrobial carried by the resilient porous applicator;
capable of receiving up to 3 cm³ of delivery fluid without escaping the resilient porous applicator; or
both.

12-13. (canceled)

14. The antimicrobial delivery device of claim 1, wherein the resilient porous applicator is loaded with from 10 mg to 200 mg of the freeze-dried antimicrobial.

15-16. (canceled)

17. The antimicrobial delivery device of claim 1, wherein:
introducing 3 cm³ of delivery fluid into the porous material from the fluid channeling body generates a 1 wt % to 5 wt % solution of antimicrobial which is carried by the porous material, wherein the weight percent is based on the freeze-dried antimicrobial after being reconstituted by the delivery fluid and excludes the weight of the porous material;
introducing 1 cm of delivery fluid into the porous material from the fluid channeling body generates a 0.5 wt % to 3 wt % solution of antimicrobial which is carried by the porous material, wherein the weight percent is based on the freeze-dried antimicrobial after being reconstituted by the delivery fluid and excludes the weight of the porous material; or
introducing 10 cm³ of delivery fluid into the porous material from the fluid channeling body generates a 2 wt % to 8 wt % solution of antimicrobial which is carried by the porous material, wherein the weight percent is based on the freeze-dried antimicrobial after being reconstituted by the delivery fluid and excludes the weight of the porous material.

18-19. (canceled)

20. The antimicrobial delivery device of claim 1, wherein the resilient porous applicator with freeze-dried antimicrobial therein prior to introduction of the delivery fluid has a total volume of about 0.2 cm³ to about 100 cm³, wherein the total volume includes both material volume and void volume defined by the material.

21-22. (canceled)

23. The antimicrobial delivery device of claim 1, wherein the fluid channeling body has a volume from 0.5 cm³ to 10 cm³.

24-25. (canceled)

26. The antimicrobial delivery device of claim 1, wherein the resilient porous applicator is a polyurethane sponge or foam.

27. The antimicrobial delivery device of claim 1, wherein the freeze-dried antimicrobial is loaded into the resilient porous applicator under vacuum pressure of less than about 100 mTorr via a sublimation period ranging from about −45° C. to less than about 10° ° C. followed by an evaporation period ranging from about 0° ° C. to about 25° C.

28. The antimicrobial delivery device of claim 27, wherein from about 70 wt % to about 90 wt % of the freeze-dried antimicrobial is loaded during the sublimation period, and about 10 wt % to about 30 wt % of the freeze-dried antimicrobial is loaded during the evaporation period.

29. The antimicrobial delivery device of claim 1, wherein the resilient porous applicator loaded with the freeze-dried antimicrobial has a moisture content of less than about 2.5 wt %.

30. (canceled)

31. The antimicrobial delivery device of claim 1, wherein a barrel of the fluid channeling body relative to the fluid channeling body attachment surface has an angle from about 30° to about 60°.

32. The antimicrobial delivery device of claim 1 wherein the fluid channeling body attachment surface is associated with a flange that extends out from the fluid outlet to provide additional surface area for attachment to the resilient porous applicator.

33. An antimicrobial delivery system, comprising:
the antimicrobial delivery device of claim 1; and
a fluid delivery device to introduce the delivery fluid into the antimicrobial delivery device.

34. The antimicrobial delivery system of claim 33, wherein the fluid delivery device is a syringe, an ampoule, a fluid pump, an automated fluid metering device, or an IV bag.

35. The antimicrobial delivery system of claim 33, wherein the fluid delivery device includes a delivery fitting adapted to connect with the channel fitting.

36. (canceled)

37. The antimicrobial delivery system of claim 33, further comprising the delivery fluid.

38. (canceled)

39. A method of preparing an antimicrobial delivery device, comprising:
loading a resilient porous material with an antimicrobial loading solution including a liquid carrier and from 0.5 wt % to 10 wt % of an antimicrobial compound;
freeze-drying the resilient porous material containing the antimicrobial loading solution to remove liquid carrier from the resilient porous material forming a resilient porous applicator loaded with a freeze-dried antimicrobial, wherein the resilient porous applicator includes a fluid-receiving surface and a skin contact surface;
attaching the fluid-receiving surface of the resilient porous applicator to a fluid channeling body attachment surface of the fluid channeling body, the fluid channeling body including (i) a fluid inlet positioned at a channel fitting that is connectable to a fluid delivery device, and (ii) a fluid outlet positioned at the fluid channeling body attachment surface to provide an opening for delivery fluid to enter the resilient porous applicator and reconstitute the antimicrobial compound within the resilient porous applicator.

40. The method of claim 39, wherein freeze-drying occurs under vacuum pressure of less than about 100 mTorr and includes a sublimation period ranging from about −45° C. to less than about 10° ° C. followed by an evaporation period ranging from about 0° ° C. to about 25° C., wherein from about 70 wt % to about 90 wt % of the freeze-dried antimicrobial is formed during the sublimation period, and about 10 wt % to about 30 wt % of the freeze-dried antimicrobial is formed during the evaporation period.

41. The method of claim 39, wherein the resilient porous applicator has a moisture content of less than about 2.5 wt %.

42. The method of claim 39, wherein the resilient porous applicator is loaded with from 10 mg to 200 mg of the freeze-dried antimicrobial.

43. The method of claim 39, wherein the channel fitting includes a barbed connector, a luer connector, a pressure fit connector, a threaded connector, a membrane, a tube, or a combination thereof.

44. The method of claim 39, further comprising sterilizing the antimicrobial delivery device and sealing the antimicrobial skin delivery device in a container.

45. The method of claim 39, wherein the resilient porous applicator includes a polymeric material, a fibrous material, or a solidified foam.

46. The method of claim 39, wherein the freeze-dried antimicrobial includes an antiseptic compound or an antibiotic compound.

47. The method of claim 39, wherein the freeze-dried antimicrobial includes an alcohol, an iodine, a chlorhexidine, a quaternary ammonium salt, an antimicrobial dye, a peroxide, a permanganate, a halogenated phenol derivative, a quinolone derivative, or a combination thereof.

48. The method of claim 39, wherein the resilient porous applicator with freeze-dried antimicrobial therein prior to introduction of the delivery fluid has a total volume of about 0.2 cm³ to about 100 cm², wherein the total volume includes both material volume and void volume defined by the material.

49. A method of delivering an antimicrobial compound to a skin surface, comprising:
connecting a delivery device to an inlet of a fluid channeling body of an antimicrobial delivery device, wherein the fluid channeling body includes a fluid outlet, and wherein the antimicrobial delivery device further includes a resilient porous applicator carrying a freeze-dried antimicrobial positioned at the fluid outlet;
flowing a delivery fluid from the delivery device through the fluid channeling body and into the resilient porous applicator to form an antimicrobial solution that includes an antimicrobial compound formed from the freeze-dried antimicrobial which is reconstituted in the delivery fluid and carried by the resilient porous applicator, and
applying the antimicrobial solution to a skin surface by applying mechanical pressure to the resilient porous applicator against the skin surface.

50. The method of claim 49, wherein connecting the delivery device to the fluid channeling body is by a pair of mated luer connectors or barbed connectors.

51. The method of claim 49, wherein the fluid channeling body is used as a handle when applying the antimicrobial solution to the skin surface.

52. The method of claim 49, wherein the antimicrobial solution formed in the resilient porous applicator includes from 0.5 wt % to 10 wt % of the antimicrobial compound.

53. (canceled)

54. The method of claim 49, wherein flowing the delivery fluid includes flowing from 1 cm³ to 20 cm³ of delivery fluid into the resilient porous applicator.

55. (canceled)

56. The method of claim 49, wherein the freeze-dried antimicrobial includes an antiseptic compound or an antibiotic compound.

57. The method of claim 49, wherein the freeze-dried antimicrobial includes an alcohol, an iodine, a chlorhexidine, a quaternary ammonium salt, an antimicrobial dye, a peroxide, a permanganate, a halogenated phenol derivative, a quinolone derivative, or a combination thereof.

58. The method of claim 49, wherein the resilient porous applicator is a sponge and the mechanical pressure includes applying a back and forth motion to the sponge against the skin surface, compressing the sponge against the skin surface, or both.
